(19) United States
(12) Patent Application Publication
(10) Pub. No.: US 2026/0114989 A1
(43) Pub. Date: Apr. 30, 2026

(54) SYSTEMS AND METHODS FOR REVERSIBLE INTRAOCULAR LENS WITH DIFFERENT SPHERICAL ABERRATION CORRECTIONS

(71) Applicant: Alcon Inc., Fribourg (CH)

(72) Inventors: Robert ANGELOPOULOS, San Jose, CA (US); Terah SMILEY, Davis, CA (US); George PETTIT, Fort Worth, TX (US); Scott CHANDLER, Menlo Park, CA (US)

(21) Appl. No.: 19/374,107

(22) Filed: Oct. 30, 2025

Related U.S. Application Data
(60) Provisional application No. 63/714,406, filed on Oct. 31, 2024.

(51) Int. Cl. A61F 2/16

(52) U.S. Cl. CPC A61F 2/1618; A61F 2/164; A61F 2240/002

(57) ABSTRACT

A reversible intraocular lens (IOL) includes an anterior optical surface and a posterior optical surface. The anterior optical surface is configured to face a cornea of a user in a first orientation. The posterior optical surface is configured to face a cornea of a user in a second orientation. The reversible IOL provides a first spherical aberration correction in the first orientation and a second spherical aberration correction in the second orientation. The first spherical aberration correction is different from the second spherical aberration correction. Advantageously the reversible IOL can provide two different spherical aberration corrections based on orientation, two different optical powers based on orientation, a variable shift in spherical aberration based on orientation, a variable shift in optical power based on orientation, an EDOF or increased visual contrast at distance vision based on orientation, correction for higher levels of corneal spherical aberration, and a reduction in photic phenomena.

### CROSS-REFERENCE TO RELATED APPLICATIONS

This application claims the benefit of U.S. Provisional Application No. 63/714,406, filed Oct. 31, 2024, which is incorporated herein by reference in its entirety.

### FIELD

The present disclosure relates to ophthalmic lens apparatuses, systems, and methods, for example, reversible intraocular lens (IOL) apparatuses, systems, and methods with different spherical aberration corrections based on orientation.

### BACKGROUND

The net optical power of the eye is determined by the optical power of the cornea, the optical power of the natural (crystalline) lens, and the distances between the cornea, the natural lens, and the retina of the human eye. The natural lens is a transparent, biconvex structure whose curvature can be changed by ciliary muscles for adjusting the optical power to allow the eye to focus on objects at different distances. This adjustment of the natural lens is known as accommodation. As a result of accommodation, spherical aberration exhibited by the natural lens shifts in the negative direction.

Spherical aberration (SA) of an optical system is a difference in optical power between light rays near the optical axis (e.g., paraxial rays) and light rays away from the optical axis (e.g., marginal rays), resulting in a blur or defocus of light. SA can have numerous causes including a spherical lens surface. The amount of SA in a lens with a spherical surface can depend upon its shape, which can be characterized by the Coddington shape factor. The SA can be minimized by shaping the lens into its best form. Also, SA can have both detrimental and beneficial optical effects, for example, degradation of the best focus (e.g., best image quality/contrast) can provide an increase in the depth of focus (e.g., extended depth of focus). Further, the SA can be minimized or eliminated by using combinations of lenses or by using aspheric lenses.

Over time, the natural lens can become less transparent in individuals suffering from cataract and/or presbyopia, e.g., due to age and/or disease, thus diminishing the amount of light that reaches the retina. A known treatment for cataract and/or presbyopia involves removing the opaque natural lens and replacing it with an IOL. The IOL can be implanted in either the anterior chamber of the eye or in the posterior chamber of the eye.

With the exception of adjustable IOLs, some current implants only provide a single SA correction, for example, targeted towards a mean distribution of corneal SA, which is not ideal for all patients, especially those that have undergone a corneal surgical correction. Also, current IOLs often have limited options for the surgeon or ophthalmologist and are not designed to provide two different levels of SA correction based on lens orientation. Further, current IOLs that provide extended depth of focus (EDOF) are often multifocal and may suffer from increased photic phenomena.

### SUMMARY

Accordingly, there is a need to develop a reversible ophthalmic lens (e.g., an IOL) that provides two different SA corrections based on orientation. Further, there is a need to develop a reversible ophthalmic lens that provides two different optical powers based on orientation. Further, there is a need to develop a reversible ophthalmic lens that provides a variable shift in SA based on orientation. Further, there is a need to develop a reversible ophthalmic lens that provides a variable shift in optical power based on orientation. Further, there is a need to develop a reversible ophthalmic lens that provides an EDOF or an increased visual contrast at distance vision based on orientation. Further, there is a need to develop a reversible ophthalmic lens that provides correction for higher levels of corneal spherical aberration (e.g., LASIK). Further, there is a need to develop a reversible ophthalmic lens that provides a reduction in photic phenomena. This novel approach can provide a customized (predetermined) reversible IOL optimized for reversibility as well as a suite of reversible IOLs each having a different difference in SA correction and/or optical power based on orientation to expand options for the surgeon, ophthalmologist, or patient.

In some aspects, a reversible ophthalmic lens may include an anterior optical surface and a posterior optical surface. In some aspects, the anterior optical surface may be configured to face a cornea of a user in a first orientation of the reversible ophthalmic lens. In some aspects, the posterior optical surface may be configured to face a cornea of a user in a second orientation of the reversible ophthalmic lens. In some aspects, the reversible ophthalmic lens may provide a first spherical aberration correction in the first orientation. In some aspects, the reversible ophthalmic lens may provide a second spherical aberration correction in the second orientation. In some aspects, the first spherical aberration correction may be different from the second spherical aberration correction.

In some aspects, the first and second spherical aberration corrections may be based at least in part on a Coddington shape factor:

$$C = \frac{{R}_{p} + {R}_{a}}{{R}_{p} - {R}_{a}}$$

where Rₐ is a radius of curvature of the anterior optical surface and Rₚ is a radius of curvature of the posterior optical surface.

In some aspects, the Coddington shape factor may be in a range from about −1.2 to about 1.2. In some aspects, the Coddington shape factor may be in a range from about −0.8 to about 0.8. In some aspects, the Coddington shape factor may be in a range from about −2.0 to about 2.0. In some aspects, the Coddington shape factor may be in a range from about −1.0 to about 1.0. In some aspects, the Coddington shape factor may be in a range from about −0.6 to about 0.6. In some aspects, the Coddington shape factor may be in a range from about −0.4 to about 0.4. In some aspects, the Coddington shape factor may be in a range from about −0.2 to about 0.2.

In some aspects, in a first orientation of the reversible ophthalmic lens, the Coddington shape factor may be greater than zero. In some aspects, in a second orientation of the reversible ophthalmic lens opposite the first orientation, the Coddington shape factor may be less than zero. In some aspects, in a first orientation of the reversible ophthalmic lens, the Coddington shape factor may be less than zero. In some aspects, in a second orientation of the reversible ophthalmic lens opposite the first orientation, the Coddington shape factor may be greater than zero. In some aspects, in a first orientation of the reversible ophthalmic lens, the Coddington shape factor may be a value in a range from about −2.0 to about 2.0 and, in a second orientation of the reversible ophthalmic lens opposite the first orientation, the Coddington shape factor may be a negative of the value in the first orientation (e.g., 0.7 in first orientation and −0.7 in second orientation, −0.5 in first orientation and 0.5 in second orientation, etc.).

In some aspects, the first and second spherical aberration corrections may be predetermined. In some aspects, the first and second spherical aberration corrections may be based at least in part on a desired optical power (e.g., 20 diopter). In some aspects, the first and second spherical aberration corrections may be based at least in part on one or more desired optical powers (e.g., 20 diopter in first orientation and 19.5 diopter in second orientation, etc.).

In some aspects, the first and second spherical aberration corrections may be based at least in part on a measured corneal spherical aberration. In some aspects, the measured corneal spherical aberration may be a mean distribution of corneal spherical aberration (e.g., about 0.27 μm to about 0.30 μm). In some aspects, the measured corneal spherical aberration may be based on one or more measured corneal spherical aberration of a user. In some aspects, the measured corneal spherical aberration may be based on one or more measured corneal spherical aberration of a population (e.g., people with cataracts, people with LASIK surgery, people with astigmatism, etc.). In some aspects, the first and second spherical aberration corrections may be based at least in part on a population distribution of measured corneal spherical aberration data.

In some aspects, in the first orientation, the first spherical aberration correction may be configured to provide a first optical power. In some aspects, in the second orientation, the second spherical aberration correction may be configured provide the first optical power.

In some aspects, in the first orientation, the first spherical aberration correction may be configured to provide an extended depth of focus (EDOF) and, in the second orientation, the second spherical aberration correction may be configured to provide a different EDOF or an increased visual contrast at distance vision.

In some aspects, in the first orientation, the first spherical aberration correction may be configured to provide a first EDOF or a first visual contrast at distance vision and, in the second orientation, the second spherical aberration correction may be configured to provide a second EDOF different from the first EDOF or a second visual contrast at distance vision different from the first visual contrast at distance vision.

In some aspects, a difference in spherical aberration between the first and second orientations may be at least about 250 nm. In some aspects, a difference in spherical aberration between the first and second orientations may be at least about 350 nm. In some aspects, a difference in spherical aberration between the first and second orientations may be at least about 100 nm. In some aspects, a difference in spherical aberration between the first and second orientations may be at least about 50 nm.

In some aspects, a difference in spherical aberration between the first and second orientations may be in a range from about −70 nm to about 350 nm. In some aspects, a difference in spherical aberration between the first and second orientations may be in a range from about −150 nm to about 0 nm. In some aspects, a difference in spherical aberration between the first and second orientations may be in a range from about 0 nm to about 350 nm. In some aspects, a difference in spherical aberration between the first and second orientations may be in a range from about −100 nm to about 100 nm. In some aspects, a difference in spherical aberration between the first and second orientations may be in a range from about −50 nm to about 50 nm.

In some aspects, a difference in optical power between the first and second orientations may be at least about 1 diopter. In some aspects, a difference in optical power between the first and second orientations may be at least about −1 diopter. In some aspects, a difference in optical power between the first and second orientations may be about 0.25 diopter. In some aspects, a difference in optical power between the first and second orientations may be in a range from about −0.8 diopter to about 0.6 diopter. In some aspects, a difference in optical power between the first and second orientations may be in a range from about −2.0 diopter to about 2.0 diopter. In some aspects, a difference in optical power between the first and second orientations may be in a range from about −1.0 diopter to about 1.0 diopter. In some aspects, a difference in optical power between the first and second orientations may be in a range from about −0.5 diopter to about 0.5 diopter. In some aspects, a difference in optical power between the first and second orientations may be in a range from about −0.25 diopter to about 0.25 diopter.

In some aspects, the anterior optical surface may include an anterior aspheric surface. In some aspects, the first spherical aberration correction may be based at least in part on the anterior aspheric surface.

In some aspects, the posterior optical surface may include a posterior aspheric surface. In some aspects, the second spherical aberration correction may be based at least in part on the posterior aspheric surface.

In some aspects, the anterior optical surface may include an anterior aspheric surface and the posterior optical surface may include a posterior aspheric surface. In some aspects, the first and second spherical aberration corrections may be based at least in part on the anterior aspheric surface and the posterior aspheric surface.

In some aspects, the anterior and posterior optical surfaces may be part of a unitary optic. In some aspects, the anterior and posterior optical surfaces may form the unitary optic. In some aspects, the unitary optic may include a monofocal lens. In some aspects, the unitary optic may include a multifocal lens. In some aspects, the unitary optic may be a biconvex lens. In some aspects, the unitary optic may be a biconcave lens. In some aspects, the unitary optic may be a positive meniscus lens. In some aspects, the unitary optic may be a negative meniscus lens.

In some aspects, the anterior optical surface may be part of a monofocal lens. In some aspects, the posterior optical surface may be part of a monofocal lens. In some aspects, the anterior optical surface may be part of a multifocal lens. In some aspects, the posterior optical surface may be part of a multifocal lens. In some aspects, the anterior optical surface may be part of a monofocal lens and the posterior optical surface may be part of a monofocal lens. In some aspects, the anterior optical surface may be part of a monofocal lens and the posterior optical surface may be part of a multifocal lens. In some aspects, the anterior optical surface may be part of a multifocal lens and the posterior optical surface may be part of a monofocal lens.

In some aspects, the anterior and posterior optical surfaces may include a flexible material, a foldable material, a shape memory material, or a combination thereof. In some aspects, the anterior and posterior optical elements may include polymethylmethacrylate (PMMA), silicone, hydrophobic acrylics, hydrophilic acrylics, collamer, copolymers (e.g., PEG-PEA/HEMA/Styrene, etc.), or a combination thereof.

In some aspects, the reversible ophthalmic lens may further include one or more haptic devices coupled to the anterior optical surface, the posterior optical surface, or both. In some aspects, the one or more haptic devices may be configured to extend in a plane of the anterior and posterior optical surfaces. In some aspects, the one or more haptic devices may include one or more vaults configured to extend out of a plane of the anterior and posterior optical surfaces. In some aspects, the first and second spherical aberration corrections may be based at least in part on a height of the one or more vaults.

In some aspects, the one or more haptic devices may be arranged symmetrically about an optical axis of the reversible ophthalmic lens. In some aspects, the one or more haptic devices may be reflection symmetric about the first and second orientations of the reversible ophthalmic lens.

In some aspects, a system may include a plurality of reversible ophthalmic lenses. In some aspects, each reversible ophthalmic lens may include an anterior optical element configured to provide a first spherical aberration correction and a posterior optical element configured to provide a second spherical aberration correction, where the first spherical aberration correction is different from the second spherical aberration correction.

In some aspects, the plurality of reversible ophthalmic lenses may include a first reversible ophthalmic lens and a second reversible ophthalmic lens. In some aspects, the first reversible ophthalmic lens may be configured to provide a first optical power. In some aspects, the second reversible ophthalmic lens may be configured to provide a second optical power different from the first optical power.

In some aspects, the reversible ophthalmic lens may include an anterior optical element and a posterior optical element. In some aspects, the anterior optical element may include the anterior optical surface. In some aspects, the posterior optical element may include the posterior optical surface. In some aspects, in the first orientation, the anterior optical element may be configured to face a cornea of a user and, in the second orientation, the posterior optical element may be configured to face a cornea of a user.

In some aspects, a difference in optical power between the first and second orientations for each reversible ophthalmic lens may be about 0.25 diopter. In some aspects, each reversible ophthalmic lens may be configured to provide a different difference in optical power between the first and second orientations in a range from about −1 diopter to about 1 diopter. In some aspects, each reversible ophthalmic lens may be configured to provide a different difference in spherical aberration between the first and second orientations in a range from about −70 nm to about 350 nm.

In some aspects, a method of designing a reversible ophthalmic lens may include selecting a desired optical power of the reversible ophthalmic lens. In some aspects, the method may further include selecting a desired first spherical aberration correction of the reversible ophthalmic lens in a first orientation in which an anterior optical element of the reversible ophthalmic lens is configured to face a cornea of a user. In some aspects, the method may further include selecting a desired second spherical aberration correction of the reversible ophthalmic lens in a second orientation in which a posterior optical element of the reversible ophthalmic lens is configured to face a cornea of a user. In some aspects, the method may further include determining a Coddington shape factor of the reversible ophthalmic lens based at least in part on the desired optical power, the desired first spherical aberration correction, and the desired second spherical aberration correction.

In some aspects, the method may further include determining one or more aspheric profiles of the reversible ophthalmic lens based at least in part on the Coddington shape factor, the desired first spherical aberration correction, and the desired second spherical aberration correction.

In some aspects, determining the Coddington shape factor may include designing the reversible ophthalmic lens, in the first orientation, to provide an EDOF. In some aspects, determining the Coddington shape factor may include designing the reversible ophthalmic lens, in the second orientation, to provide an increased visual contrast at distance vision.

In some aspects, the method may further include determining a corneal spherical aberration based on one or more measured corneal spherical aberrations of a user or of a population.

In some aspects, a reversible ophthalmic lens may include an anterior optical surface and a posterior optical surface. In some aspects, in a first orientation, the anterior optical surface may be configured to face a cornea of a user and the reversible ophthalmic lens may be configured to provide a first spherical aberration correction. In some aspects, in a second orientation, the posterior optical surface may be configured to face a cornea of a user and the reversible ophthalmic lens may be configured to provide a second spherical aberration correction. In some aspects, the first spherical aberration correction produced in the first orientation may be different from the second spherical aberration correction produced in the second orientation.

In some aspects, in the first orientation, the reversible ophthalmic lens may be configured to provide a first optical power. In some aspects, in the second orientation, the reversible ophthalmic lens may be configured to provide a second optical power. In some aspects, the first optical power produced in the first orientation may be different from the second optical power produced in the second orientation.

In some aspects, in the first orientation, the reversible ophthalmic lens may be configured to provide a first spherical aberration correction and a first optical power. In some aspects, in the second orientation, the reversible ophthalmic lens may be configured to provide a second spherical aberration correction and a second optical power. In some aspects, the first spherical aberration correction produced in the first orientation may be different from the second spherical aberration correction produced in the second orientation, and the first optical power produced in the first orientation may be different from the second optical power produced in the second orientation.

In some aspects, the first spherical aberration correction produced in the first orientation may be different from the second spherical aberration correction produced in the second orientation, and the first optical power produced in the first orientation may be substantially similar to the second optical power produced in the second orientation. In some aspects, the first spherical aberration correction produced in the first orientation may be substantially similar to the second spherical aberration correction produced in the second orientation, and the first optical power produced in the first orientation may be different from the second optical power produced in the second orientation.

In some aspects, a reversible ophthalmic lens may include an anterior optical surface configured to face a cornea of a user in a first orientation of the reversible ophthalmic lens, and a posterior optical surface configured to face a cornea of a user in a second orientation of the reversible ophthalmic lens. In some aspects, the reversible ophthalmic lens provides a first optical power in the first orientation and a second optical power in the second orientation. In some aspects, the first optical power is different from the second optical power by a desired shift in optical power.

In some aspects, the desired shift in optical power, first optical power, and second optical power are based at least in part on a Coddington shape factor:

$$C = \frac{{R}_{p} + {R}_{a}}{{R}_{p} - {R}_{a}}$$

where Rₐ is a radius of curvature of the anterior optical surface and Rₚ is a radius of curvature of the posterior optical surface. In some aspects, the Coddington shape factor is in a range from −1.2 to 1.2.

In some aspects, the first and second optical powers are predetermined and based at least in part on the desired shift in optical power. In some aspects, the desired shift in optical power between the first and second orientations is at least 1 diopter. In some aspects, the desired shift in optical power between the first and second orientations is 0.5 diopter. In some aspects, the desired shift in optical power between the first and second orientations is 0.25 diopter. In some aspects, the desired shift in optical power between the first and second orientations is in a range from −0.8 diopter to 0.6 diopter.

In some aspects, the anterior and posterior optical surfaces are part of a unitary optic. In some aspects, the anterior optical surface is part of a monofocal lens, and the posterior optical surface is part of a monofocal lens. In some aspects, the anterior optical surface is part of a multifocal lens, and the posterior optical surface is part of a multifocal lens.

In some aspects, the anterior and posterior optical surfaces include a flexible material, a foldable material, a shape memory material, or a combination thereof.

In some aspects, the reversible ophthalmic lens further includes one or more haptic devices coupled to the anterior optical surface, the posterior optical surface, or both.

In some aspects, a system includes a plurality of reversible ophthalmic lenses. In some aspects, each reversible ophthalmic lens is a reversible ophthalmic lens that includes an anterior optical surface configured to face a cornea of a user in a first orientation of the reversible ophthalmic lens, a posterior optical surface configured to face a cornea of a user in a second orientation of the reversible ophthalmic lens, a first optical power in the first orientation, a second optical power in the second orientation, and the first optical power is different from the second optical power by a desired shift in optical power.

In some aspects, the plurality of reversible ophthalmic lens includes a first reversible ophthalmic lens and a second reversible ophthalmic lens. In some aspects, the first reversible ophthalmic lens is configured to provide a first optical power, and the second reversible ophthalmic lens is configured to provide a second optical power different from the first optical power. In some aspects, a difference in optical power between the first and second orientations for each reversible ophthalmic lens is 0.25 diopter. In some aspects, each reversible ophthalmic lens is configured to provide a different difference in optical power between the first and second orientations in a range from −1 diopter to 1 diopter.

In some aspects, a method of designing a reversible ophthalmic lens may include selecting a desired shift in optical power of the reversible ophthalmic lens. In some aspects, the method may further include selecting a desired first optical power of the reversible ophthalmic lens in a first orientation in which an anterior optical element of the reversible ophthalmic lens is configured to face a cornea of a user. In some aspects, the method may further include selecting a desired second optical power of the reversible ophthalmic lens in a second orientation in which a posterior optical element of the reversible ophthalmic lens is configured to face a cornea of a user. In some aspects, the method may further include determining a Coddington shape factor of the reversible ophthalmic lens based at least in part on the desired shift in optical power, the desired first optical power, and the desired second spherical optical power.

In some aspects, the method may further include determining one or more aspheric profiles of the reversible ophthalmic lens based at least in part on the Coddington shape factor and the desired shift in optical power.

In some aspects, the determining the Coddington shape factor comprises designing the reversible ophthalmic lens, in the first orientation, to provide an extended depth of focus (EDOF). In some aspects, the determining the Coddington shape factor comprises designing the reversible ophthalmic lens, in the second orientation, to provide an increased visual contrast at distance vision.

Implementations of any of the techniques described above may include a system, a method, a process, a device, and/or an apparatus. The details of one or more implementations are set forth in the accompanying drawings and the description below. Other features will be apparent from the description and drawings, and from the claims.

Further features and example aspects of the present disclosure, as well as the structure and operation of various aspects, are described in detail below with reference to the accompanying drawings. It is noted that the aspects are not limited to the specific aspects described herein. Such aspects are presented herein for illustrative purposes only. Additional aspects will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the aspects and, together with the description, further serve to explain the principles of the aspects and to enable a person skilled in the relevant art(s) to make and use the aspects.

FIG. 1 is a schematic top illustration of a reversible intraocular lens (IOL) in a first orientation, according to an example aspect.

FIG. 1A is a schematic side illustration of the reversible IOL shown in FIG. 1.

FIG. 2 is a schematic top illustration of the reversible IOL in a second orientation, according to an example aspect.

FIG. 2A is a schematic side illustration of the reversible IOL shown in FIG. 2.

FIG. 3 is a schematic side illustration of a system with an implanted reversible IOL in the first orientation, according to an example aspect.

FIG. 4 is a schematic side illustration of the system with the implanted reversible IOL in the second orientation, according to an example aspect.

FIG. 5 is a schematic top illustration of a reversible IOL with symmetric haptic devices and one or more aspheric surfaces, according to example aspects.

FIG. 5A is a schematic side illustration of the reversible IOL shown in FIG. 5.

FIG. 6 shows a plot of shifts in spherical aberration between first and second orientations of a reversible IOL as a function of the Coddington shape factor, according to an example aspect.

FIG. 7 shows a plot of shifts in optical power between first and second orientations of a reversible IOL as a function of the Coddington shape factor, according to an example aspect.

FIG. 8 illustrates a flow diagram for designing a reversible IOL, according to an example aspect.

The features and example aspects of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

Provided herein are system, apparatus, device, method, and/or computer program product aspects, and/or combinations and sub-combinations thereof, for reversible ophthalmic lenses, such as IOLs with different spherical aberration corrections based on orientation.

A reversible IOL as described below may have a first spherical aberration correction in a first orientation (forward) and a second spherical aberration correction in a reversed second orientation (backward) that is different than the first spherical aberration correction to provide variable shifts in spherical aberration based on orientation (forward or backward) of the reversible IOL.

The present disclosure is generally directed to a reversible ophthalmic lens (e.g., an IOL, or contact lens) having an optical profile, such as a surface profile or Coddington shape factor, that produces a shift in spherical aberration correction and/or a shift in optical power based on the orientation of the reversible ophthalmic lens. In the following description, lens features (e.g., shape) providing a reversible shift in spherical aberration correction and/or a reversible shift in optical power are described in connection with IOLs. However, the present disclosure contemplates that those lens features may also be applied to other ophthalmic lenses, for example, but not limited to, contact lenses, spectacles, eyeglasses, or any other lenses designed to augment vision.

This specification discloses one or more aspects that incorporate the features of this present disclosure.

The aspect(s) described, and references in the specification to “one aspect,” “an aspect,” “an example aspect,” “an exemplary aspect,” etc., indicate that the aspect(s) described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to effect such feature, structure, or characteristic in connection with other aspects whether or not explicitly described.

Spatially relative terms, such as “beneath,” “below,” “lower,” “above,” “on,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Numerical values, including endpoints of ranges, can be expressed herein as approximations preceded by the term “about,” “substantially,” “approximately,” or the like. In such cases, other aspects include the particular numerical value. Regardless of whether a numerical value is expressed as an approximation, two aspects are included in this disclosure: one expressed as an approximation, and another not expressed as an approximation. It will be further understood that an endpoint of each range is significant both in relation to another endpoint, and independently of another endpoint.

Aspects of the disclosure may be implemented in hardware, firmware, software, or any combination thereof. Aspects of the disclosure may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

The term “spherical aberration” or “SA” as used herein indicates a difference in optical power for light rays near the optical axis (e.g., paraxial rays) and light rays away from the optical axis (e.g., marginal rays) of an optical system. For lenses with spherical surfaces, light rays further from the optical axis (e.g., marginal rays) are refracted more than rays closer to the optical axis (e.g., paraxial rays) resulting in different focal points. Rays that are parallel to the optical axis but at different distances away from the optical axis (e.g., marginal rays versus paraxial rays) do not converge at the same focal point, resulting in longitudinal and transverse SA due to the different focal points (e.g., marginal ray focus and paraxial ray focus). The distance between the marginal ray focus and the paraxial ray focus along the optical axis is the longitudinal SA (LSA), and the distance of the marginal rays from the optical axis at the paraxial ray focus is the transverse SA (TSA). In some aspects, the SA can be defined as the LSA. In some aspects, the SA can be defined as the “circle of least confusion,” which is the aberrated spot diameter located between the marginal ray focus and the paraxial ray focus along the optical axis at an intersection of marginal rays and paraxial rays.

The term “spherical aberration correction” or “SA correction” as used herein indicates a correction or accommodation of a spherical aberration of an optical system. In some aspects, SA correction can be performed to correct or accommodate a corneal spherical aberration by changing the Coddington shape factor of a reversible IOL. In some aspects, SA correction can be performed to correct or accommodate a corneal spherical aberration by changing one or more aspheric surfaces of a reversible IOL.

The term “Coddington shape factor” or “shape factor” as used herein indicates a numerical quantity characterizing a shape of a lens based on radii of curvature and relates to the distribution of the total optical power, which can vary between the anterior surface and the posterior surface of the lens. The Coddington shape factor is unitless and can characterize the degree of bending of the lens. The shape of a lens can be characterized by the Coddington shape factor:

$$C = \frac{{R}_{p} + {R}_{a}}{{R}_{p} - {R}_{a}}$$

where Rₐ is a radius of curvature of the anterior lens surface and Rₚ is a radius of curvature of the posterior lens surface. In some aspects, the Coddington shape factor for a reversible IOL in a first orientation (forward) is:

$${C}_{1} = \frac{{R}_{p} + {R}_{a}}{{R}_{p} - {R}_{a}}$$

where Rₐ is a radius of curvature of the anterior optical surface and Rₚ is a radius of curvature of the posterior optical surface. In some aspects, the Coddington shape factor for a reversible IOL in a reversed second orientation (backward) is:

$${C}_{2} = \frac{{R}_{a} + {R}_{p}}{{R}_{a} - {R}_{p}} = - {C}_{1}$$

where Rₐ is a radius of curvature of the anterior optical surface and Rₚ is a radius of curvature of the posterior optical surface.

The term “extended depth of focus” or “EDOF” as used herein indicates a lens or multiple lenses that create a single, contiguous, elongated focal point that enhances depth of focus. In some aspects, EDOF can provide a sharp focus over a wide range of distances (e.g., near vision, intermediate vision, distance vision).

The term “distance vision” as used herein indicates vision for objects that are at a calibrated distance from the viewer, for example, at least 20 feet or more from the viewer. Distance vision is for objects farther than intermediate vision.

The term “intermediate vision” as used herein indicates vision for objects that are at a calibrated distance from the viewer, for example, between 18 inches and 20 feet from the viewer. Intermediate vision is for objects farther than near vision.

The term “near vision” as used herein indicates vision for objects that are at a calibrated distance from the viewer, for example, 18 inches or less from the viewer. Near vision is for objects closer than intermediate vision.

The term “aspheric surface” or “aspheric profile” or “aspheric equation” as used herein indicates a lens whose surface is not a portion of a sphere or cylinder and that can be characterized by:

$$z ( r ) = \frac{{r}^{2}}{R ( 1 + \sqrt{1 - ( 1 + κ ) \frac{{r}^{2}}{{R}^{2}}} )} + {α}_{4} {r}^{4} + {α}_{6} {r}^{6} + …$$

where the optical axis is in the z-direction, z(r) is the sag or z-component of the displacement of the surface from the vertex at a distance r from the optical axis, R is the radius of curvature, κ is the conic constant as measured at the vertex (r=0), and coefficients αᵢ represent the deviation of the surface from the axially symmetric quadric surface specified by R and κ. In some aspects, the coefficients αᵢ (e.g., 4^{th}-order coefficient α₄, 6^{th}-order coefficient α₆, etc.) can be tuned or adjusted (e.g., optimized) to minimize or eliminate SA of the lens. In some aspects, one or more of the coefficients αᵢ and/or the conic constant κ may be zero.

### Example Reversible IOLs

As discussed above, the net optical power of the eye is determined by the optical power of the cornea, the optical power of the natural lens, and the distances between the cornea, the natural lens, and the retina of the human eye. The human eye is a generally spherical body defined by an outer wall called the sclera, having a transparent bulbous front portion called the cornea. The natural lens is located within the generally spherical body behind the cornea. The cornea contributes most of the eye's optical power, but its focus is fixed. The natural lens is a transparent, biconvex structure whose curvature can be changed by ciliary muscles for adjusting the optical power to allow the eye to focus on objects at different distances. This adjustment of the natural lens is known as accommodation. As a result of accommodation, spherical aberration exhibited by the natural lens shifts in the negative direction.

SA of an optical system is a difference in optical power between light rays near the optical axis (e.g., paraxial rays) and light rays away from the optical axis (e.g., marginal rays), resulting in a blur or defocus of light. For lenses with spherical surfaces, rays that are parallel to the optical axis but at different distances away from the optical axis (e.g., marginal rays versus paraxial rays) do not converge at the same focal point, resulting in longitudinal SA (LSA). For a single lens, SA can be minimized by shaping the lens into its best form. The amount of SA in a lens with spherical surfaces depends upon its shape. The shape of a lens can be characterized by the Coddington shape factor:

$$C = \frac{{R}_{p} + {R}_{a}}{{R}_{p} - {R}_{a}}$$

where Rₐ is a radius of curvature of the anterior lens surface and Rₚ is a radius of curvature of the posterior lens surface.

Further, SA can be minimized or eliminated by using combinations of lenses or by using aspheric lenses. An aspheric lens is a lens whose surface profile is not a portion of a sphere or cylinder and that can be characterized by an aspheric profile:

$$z ( r ) = \frac{{r}^{2}}{R ( 1 + \sqrt{1 - ( 1 + κ ) \frac{{r}^{2}}{{R}^{2}}} )} + {α}_{4} {r}^{4} + {α}_{6} {r}^{6} + …$$

where the optical axis is in the z-direction, z(r) is the sag or z-component of the displacement of the surface from the vertex at a distance r from the optical axis, R is the radius of curvature, κ is the conic constant as measured at the vertex (r=0), and coefficients ai represent the deviation of the surface from the axially symmetric quadric surface specified by R and κ. The coefficients αᵢ (e.g., 4^{th}-order coefficient α₄, 6^{th}-order coefficient α₆, etc.) can be tuned or optimized to minimize or eliminate SA of the lens.

Over time, the natural lens can become less transparent in individuals suffering from cataract, e.g., due to age and/or disease, thus diminishing the amount of light that reaches the retina. A known treatment for cataract involves removing the opaque natural lens and replacing it with an artificial IOL. The IOL can be implanted in either the anterior chamber of the eye or in the posterior chamber of the eye.

Currently, some IOLs only provide a single SA correction, for example, targeted towards a mean distribution of corneal SA (e.g., about 0.27 μm to about 0.30 μm), which is not ideal for all patients, especially those that have undergone a corneal surgical correction (e.g., LASIK). Also, current IOLs have limited options for the surgeon or ophthalmologist and are not designed to provide two different levels of SA correction based on lens orientation. Further, current IOLs that provide EDOF are multifocal and can create photic phenomena (e.g., halos, glare, artifacts, etc.).

Aspects of reversible IOL apparatuses, systems, and methods as discussed below can provide two different SA corrections based on orientation, two different optical powers based on orientation, a variable shift in SA based on orientation, a variable shift in optical power based on orientation, an EDOF or an increased visual contrast at distance vision based on orientation, correction for higher levels of corneal SA (e.g., LASIK), and a reduction in photic phenomena.

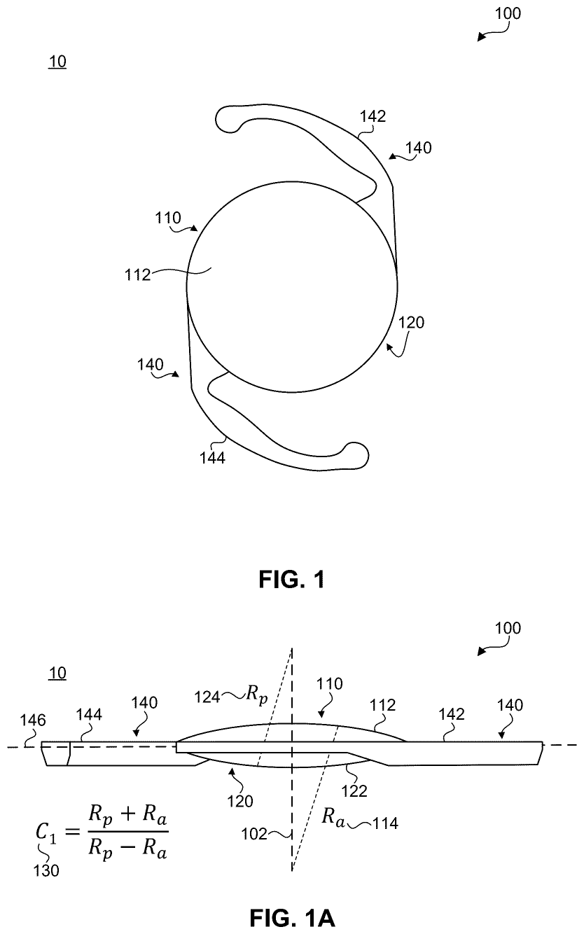

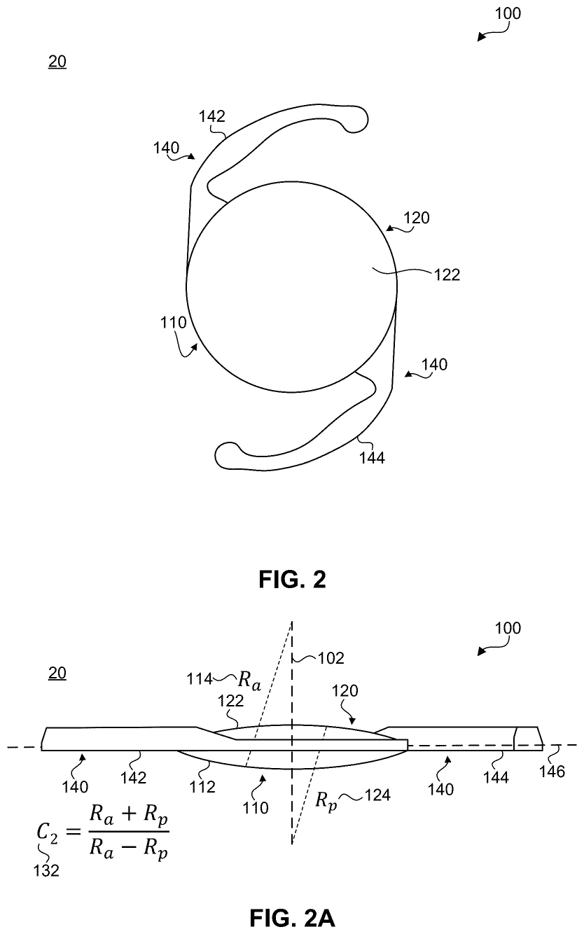

FIGS. 1 and 1A illustrate reversible IOL 100 in a first orientation 10 (forward), according to various example aspects. FIGS. 2 and 2A illustrate reversible IOL 100 in a second orientation 20 (backward), according to various example aspects. Reversible IOL 100 can be configured to be reversible and provide two different SA corrections based on orientation (e.g., forward, backward). Reversible IOL 100 can be further configured to provide two different optical powers based on orientation. Reversible IOL 100 can be further configured to provide a variable shift in SA based on orientation. Reversible IOL 100 can be further configured to provide a variable shift in optical power based on orientation. Reversible IOL 100 can be further configured to provide an EDOF or an increased visual contrast at distance vision based on orientation. Reversible IOL 100 can be further configured to provide correction for higher levels of corneal SA (e.g., LASIK). Reversible IOL 100 can be further configured to provide a reduction in photic phenomena (e.g., halos, glare, artifacts, etc.).

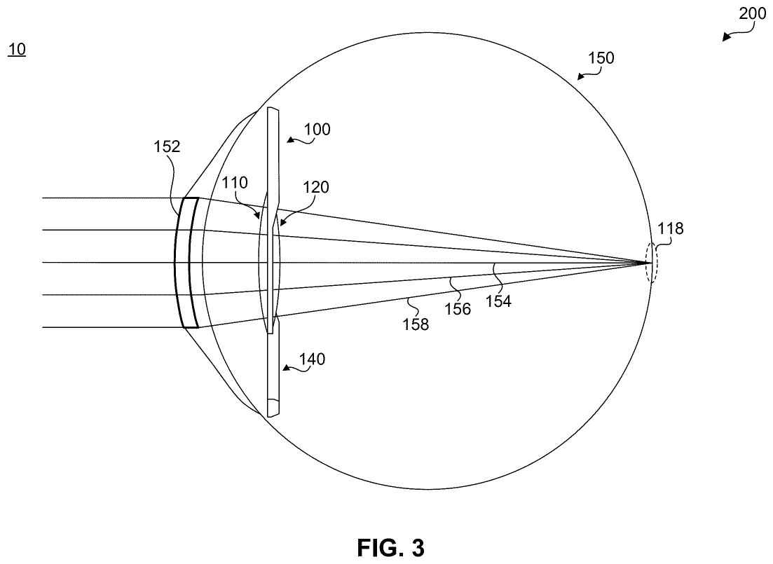

Although reversible IOL 100 is shown in FIGS. 1, 1A, 2, and 2A as a stand-alone apparatus and/or system, the aspects of this disclosure can be used with other apparatuses, systems, and/or methods, such as, but not limited to, elements in FIGS. 3-5, 5A, and 6-8, e.g., system 200, reversible IOL 100′, SA shift function 610, optical power shift function 710, and/or flow diagram 800.

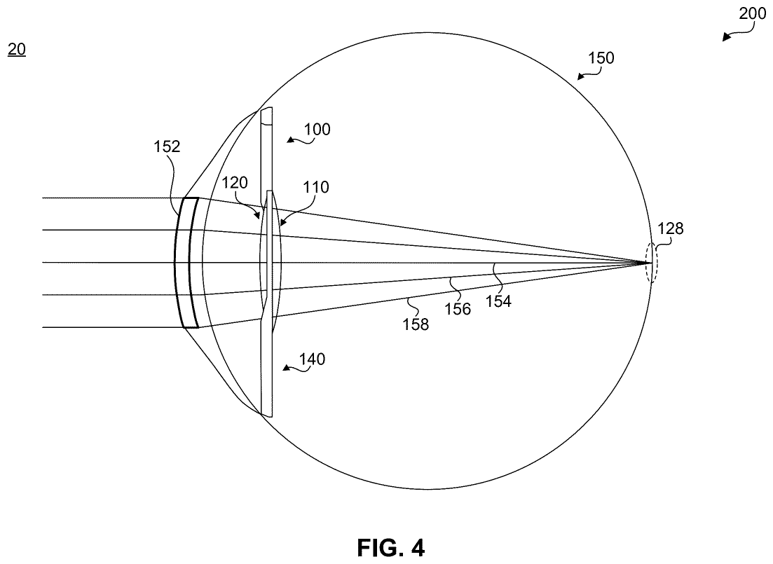

As shown in FIGS. 1, 1A, 2, and 2A, reversible IOL 100 can include optical axis 102, anterior optical element 110, posterior optical element 120, and haptic device 140. In some aspects, reversible IOL 100 can provide a first SA correction in first orientation 10 (e.g., first SA correction 118 (FIG. 3)) and a second SA correction in second orientation 20 (e.g., second SA correction 128 (FIG. 4)). In some aspects, reversible IOL 100 can provide a first optical power in first orientation 10 (e.g., 20 diopter) and a second optical power in second orientation 20 (e.g., 21 diopter). In some aspects, reversible IOL 100 can provide a variable shift in SA (e.g., decrease or increase SA) between first orientation 10 and second orientation 20. In some aspects, reversible IOL 100 can provide a variable shift in optical power (e.g., decrease or increase optical power) between first orientation 10 and second orientation 20.

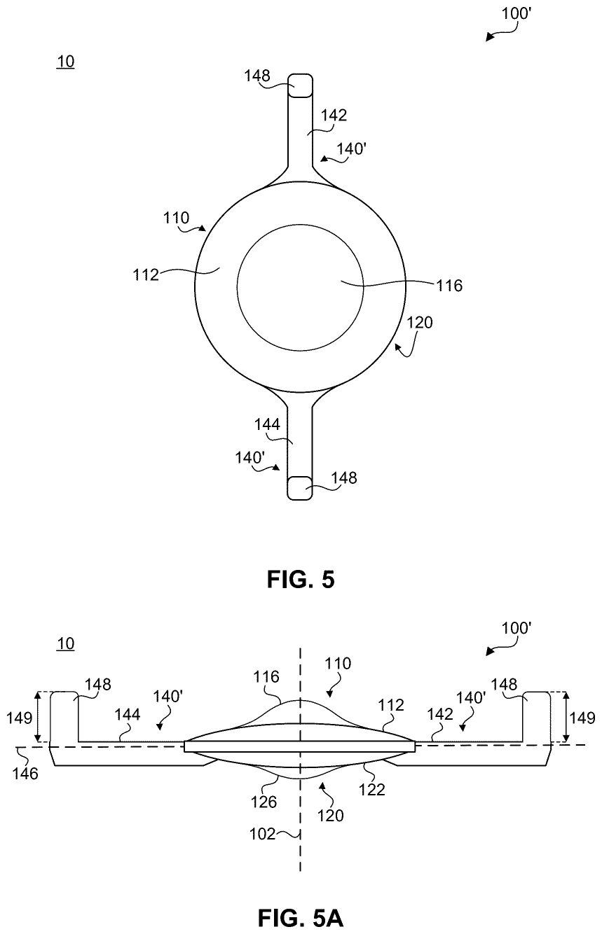

Anterior optical element 110 can be configured to provide a first SA correction (e.g., first SA correction 118 (FIG. 3)). Anterior optical element 110 can be further configured to provide a first optical power. As shown in FIGS. 1, 1A, 2, and 2A, anterior optical element 110 can include anterior surface 112 and anterior radius of curvature Rₐ 114. In some aspects, anterior surface 112 can include an aspheric profile. For example, as shown in FIGS. 5 and 5A, anterior optical element 110 can include anterior aspheric surface 116. In some aspects, the first SA correction (e.g., first SA correction 118 (FIG. 3)) can be based at least in part on anterior aspheric surface 116.

Posterior optical element 120 can be configured to provide a second SA correction (e.g., second SA correction 128 (FIG. 4)). Posterior optical element 120 can be further configured to provide a second optical power. In some aspects, the first SA correction can be different from the second SA correction. In some aspects, the first optical power can be different from the second optical power. As shown in FIGS. 1, 1A, 2, and 2A, posterior optical element 120 can include posterior surface 122 and posterior radius of curvature Rₚ 124. In some aspects, posterior surface 122 can include an aspheric profile. For example, as shown in FIGS. 5 and 5A, posterior optical element 120 can include posterior aspheric surface 126. In some aspects, the second SA correction (e.g., second SA correction 128 (FIG. 4)) can be based at least in part on posterior aspheric surface 126.

In some aspects, anterior optical element 110 can include anterior aspheric surface 116 and posterior optical element 120 can include posterior aspheric surface 126. In some aspects, the first and second SA corrections (e.g., first and second SA corrections 118, 128) can be based at least in part on anterior aspheric surface 116 and posterior aspheric surface 126.

In some aspects, as shown in FIG. 1A, in first orientation 10 (forward), reversible IOL 100 can have a first Coddington shape factor 130:

$${C}_{1} = \frac{{R}_{p} + {R}_{a}}{{R}_{p} - {R}_{a}}$$

where Rₐ is the anterior radius of curvature Rₐ 114 of anterior surface 112 and Rₚ is the posterior radius of curvature Rₚ 124 of posterior surface 122.

In some aspects, as shown in FIG. 2A, in second orientation 20 (backward), reversible IOL 100 can have a second Coddington shape factor 132:

$${C}_{2} = \frac{{R}_{a} + {R}_{p}}{{R}_{a} - {R}_{p}} = - {C}_{1}$$

where Rₐ is the anterior radius of curvature Rₐ 114 of anterior surface 112 and Rₚ is the posterior radius of curvature Rₚ 124 of posterior surface 122.

In some aspects, first Coddington shape factor 130 (e.g., C₁=1.2) of reversible IOL 100 in first orientation 10 can be the negative of second Coddington shape factor 132 (e.g., C₂=−1.2=−C₁) of reversible IOL 100 in second orientation 20. In some aspects, second Coddington shape factor 132 (e.g., C₂=0.8) of reversible IOL 100 in second orientation 20 can be the negative of first Coddington shape factor 130 (e.g., C₁=−0.8=−C₂) of reversible IOL 100 in first orientation 10.

In some aspects, anterior and posterior optical elements 110, 120 can form a unitary optic (e.g., reversible IOL 100). In some aspects, the unitary optic can have anterior surface 112 with anterior radius of curvature Rₐ 114 and posterior surface 122 with posterior radius of curvature Rₚ 124. In some aspects, the unitary optic can include a monofocal lens. In some aspects, the unitary optic can include a multifocal lens. In some aspects, the unitary optic can be a biconvex lens. In some aspects, the unitary optic can be a biconcave lens. In some aspects, the unitary optic can be a positive meniscus lens. In some aspects, the unitary optic can be a negative meniscus lens.

In some aspects, anterior optical element 110 can include a monofocal lens. In some aspects, anterior optical element 110 can include a multifocal lens. In some aspects, posterior optical element 120 can include a monofocal lens. In some aspects, posterior optical element 120 can include a multifocal lens.

In some aspects, anterior optical element 110 can include a monofocal lens and posterior optical element 110 can include a monofocal lens. In some aspects, anterior optical element 110 can include a monofocal lens and posterior optical element 120 can include a multifocal lens. In some aspects, anterior optical element 110 can include a multifocal lens and posterior optical element 120 can include a monofocal lens.

In some aspects, anterior and posterior optical elements 110, 120 can include a flexible material, a foldable material, a shape memory material, or a combination thereof. In some aspects, anterior and posterior optical elements 110, 120 can include polymethylmethacrylate (PMMA), silicone, hydrophobic acrylics, hydrophilic acrylics, collamer, copolymers (e.g., polyethylene glycol (PEG)-phenyl ether acrylate (PEA)/hydroxyethylmethacrylate (HEMA)/Styrene, etc.), or a combination thereof.

Haptic device 140 can be configured to secure and stabilize reversible IOL 100 in place when implanted in an eye. Haptic device 140 can be further configured to be arranged symmetrically about optical axis 102 of reversible IOL 100 such that there is no change (e.g., reflection symmetric) of haptic device 140 between first orientation 10 and second orientation 20. As shown in FIGS. 1, 1A, 2, and 2A, haptic device 140 can include first haptic arm 142 and second haptic arm 144. In some aspects, reversible IOL 100 can omit haptic device 140. In some aspects, haptic device 140 can include a plurality of haptic arms.

In some aspects, haptic device 140 can be coupled to anterior optical element 110, posterior optical element 120, or both. In some aspects, haptic device 140 can be configured to extend in a plane of anterior and posterior optical elements 110, 120. For example, as shown in FIGS. 1A and 2A, first and second haptic arms 142, 144 can extend in plane 146 of anterior and posterior optical elements 110, 120. In some aspects, haptic device 140 can be arranged symmetrically about optical axis 102 of reversible IOL 100. For example, as shown in FIGS. 5 and 5A, first and second haptic arms 142, 144 of haptic device 140′ can be arranged symmetrically about optical axis 102 of reversible IOL 100 such that there is no change (e.g., reflection symmetric) between first orientation 10 and second orientation 20.

In some aspects, haptic device 140 can include one or more vaults. For example, as shown in FIGS. 5 and 5A, first and second haptic arms 142, 144 of haptic device 140′ can include vaults 148 extending out of plane 146 of anterior and posterior optical elements 110, 120. In some aspects, first and second SA corrections (e.g., first and second SA corrections 118, 128) can be based at least in part on a height of vaults 148. For example, as shown in FIG. 5A, vaults 148 can have height 149 extending away from plane 146 of anterior and posterior optical elements 110, 120 toward anterior optical element 110 in first orientation 10. In some aspects, vaults 148 can extend away from plane 146 of anterior and posterior optical elements 110, 120 toward posterior optical element 120 in first orientation 10.

FIG. 3 illustrates system 200 with reversible IOL 100 implanted in first orientation 10, according to an example aspect. FIG. 4 illustrates system 200 with reversible IOL 100 implanted in second orientation 20, according to an example aspect. Although system 200 is shown in FIGS. 3 and 4 as a stand-alone apparatus and/or system, the aspects of this disclosure can be used with other apparatuses, systems, and/or methods, such as, but not limited to, elements in FIGS. 1, 1A, 2, 2A, 5, 5A, and 6-8, e.g., reversible IOL 100, reversible IOL 100′, SA shift function 610, optical power shift function 710, and/or flow diagram 800.

As shown in FIGS. 3 and 4, system 200 can include reversible IOL 100, eye 150, cornea 152, optical axis 154, paraxial rays 156, and marginal rays 158. In some aspects, anterior optical element 110 of reversible IOL 100 can face cornea 152 in first orientation 10. In some aspects, posterior optical element 120 of reversible IOL 100 can face cornea 152 in second orientation 20. As shown in FIG. 3, reversible IOL 100 can be implanted within eye 150 in first orientation 10, so that anterior optical element 110 faces cornea 152, thereby providing first SA correction 118. As shown in FIG. 4, reversible IOL 100 can be implanted within eye 150 in second orientation 20, so that posterior optical element 120 faces cornea 152, thereby providing second SA correction 128. In some aspects, first SA correction 118 can be different from second SA correction 128.

In some aspects, first and second SA corrections 118, 128 can be predetermined. For example, first and second SA corrections 118, 128 can be based at least in part on a desired optical power, a measured corneal SA (e.g., LASIK), a Coddington shape factor, one or more aspheric profiles, or a combination thereof.

In some aspects, first and second SA corrections 118, 128 can be determined based on an optimization model or optimization algorithm to optimize a set of parameters of reversible IOL 100. In some aspects, the parameters can include the optical power in first orientation 10, the optical power in second orientation 20, the Coddington shape factor in first orientation 10 (e.g., first Coddington shape factor 130), the Coddington shape factor in second orientation 20 (e.g., second Coddington shape factor 132), the anterior aspheric profile (e.g., anterior aspheric surface 116), the posterior aspheric profile (e.g., posterior aspheric surface 126), the focal length of anterior optical element 110, the focal length of posterior optical element 120, the anterior radius of curvature Rₐ 114 of anterior surface 112, the posterior radius of curvature Rₚ 124 of posterior surface 122, a height (vault) of haptic device 140 in first orientation 10, a height (vault) of haptic device 140 in second orientation 20, or a combination thereof.

In some aspects, a model of reversible IOL 100 (e.g., ZEMAX) based on the set of parameters can be defined. In some aspects, an inverse optimization of the set of parameters can be performed such that the model of reversible IOL 100 produces a desired first SA correction (e.g., first SA correction 118) in first orientation 10 and a desired second SA correction (e.g., second SA correction 128) in second orientation 20. In some aspects, an inverse optimization of the set of parameters can be performed such that the model of reversible IOL 100 produces a desired first optical power in first orientation 10 and a desired second optical power in second orientation 20. In some aspects, the optimization model or optimization algorithm can include simulated annealing, gradient descent, finite difference, interpolation, population models, regression, parameter adaptation, supervised machine learning, unsupervised machine learning, neural networks, classification models, clustering, vector quantization, stochastic gradient descent, implicit updates, leaky averaging, momentum methods, adaptive gradient (AdaGrad), backpropagation, root mean square propagation (RMSProp), adaptive moment estimation (Adam), or a combination thereof. In some aspects, the inverse optimization can be performed by an optimization algorithm.

In some aspects, first and second SA corrections 118, 128 can be based at least in part on a desired optical power (e.g., 20 diopter). In some aspects, first and second SA corrections 118, 128 can be based at least in part on one or more desired optical powers (e.g., 20 diopter in first orientation 10 and 19.5 diopter in second orientation 20, etc.).

In some aspects, first and second SA corrections 118, 128 can be based at least in part on a measured corneal SA. In some aspects, the measured corneal SA can be a mean distribution of corneal SA (e.g., about 0.27 μm to about 0.30 μm). In some aspects, the measured corneal SA can be based on one or more measured corneal SA of a user. In some aspects, the measured corneal SA can be based on one or more measured corneal SA of a population (e.g., people with cataracts, people with LASIK surgery, people with astigmatism, etc.).

In some aspects, first and second SA corrections 118, 128 can be based at least in part on a Coddington shape factor:

$$C = \frac{{R}_{p} + {R}_{a}}{{R}_{p} - {R}_{a}}$$

where Rₐ is the anterior radius of curvature Rₐ 114 of anterior surface 112 and Rₚ is the posterior radius of curvature Rₚ 124 of posterior surface 122.

In some aspects, the Coddington shape factor of reversible IOL 100 can be in a range from about −1.2 to about 1.2. In some aspects, the Coddington shape factor of reversible IOL 100 can be in a range from about −0.8 to about 0.8. In some aspects, the Coddington shape factor of reversible IOL 100 can be in a range from about −2.0 to about 2.0. In some aspects, the Coddington shape factor of reversible IOL 100 can be in a range from about −1.0 to about 1.0. In some aspects, the Coddington shape factor of reversible IOL 100 can be in a range from about −0.6 to about 0.6. In some aspects, the Coddington shape factor of reversible IOL 100 can be in a range from about −0.4 to about 0.4. In some aspects, the Coddington shape factor of reversible IOL 100 can be in a range from about −0.2 to about 0.2.

In some aspects, in first orientation 10 (forward), the first Coddington shape factor 130 can be greater than zero. In some aspects, in second orientation 20 (backward), the second Coddington shape factor 132 can be less than zero. In some aspects, in first orientation 10 (forward), the first Coddington shape factor 130 can be less than zero. In some aspects, in second orientation 20 (backward), the second Coddington shape factor 132 can be greater than zero. In some aspects, in first orientation 10 (forward), the first Coddington shape factor 130 can be a value in a range from about −2.0 to about 2.0 and, in second orientation 20 (backward), the second Coddington shape factor 132 can be a negative of the value in first orientation 10 (e.g., 0.7 in first orientation 10 and −0.7 in second orientation 20, −0.5 in first orientation 10 and 0.5 in second orientation 20, etc.).

In some aspects, as shown in FIG. 3, in first orientation 10, anterior optical element 110 can be configured to face cornea 152 of a user. In some aspects, as shown in FIG. 4, in second orientation 20, posterior optical element 120 can be configured to face cornea 152 of a user.

In some aspects, in first orientation 10, first SA correction 118 can be configured to provide an EDOF and, in second orientation 20, second SA correction 128 can be configured to provide a different EDOF or an increased visual contrast at distance vision.

In some aspects, in first orientation 10, first SA correction 118 can be configured to provide a first EDOF or a first visual contrast at distance vision and, in second orientation 20, second SA correction 128 can be configured to provide a second EDOF different from the first EDOF or a second visual contrast at distance vision different from the first visual contrast at distance vision.

In some aspects, a difference in SA between first and second orientations 10, 20 can be at least about 250 nm. In some aspects, a difference in SA between first and second orientations 10, 20 can be at least about 350 nm. In some aspects, a difference in SA between first and second orientations 10, 20 can be at least about 100 nm. In some aspects, a difference in SA between first and second orientations 10, 20 can be at least about 50 nm.

In some aspects, a difference in SA between first and second orientations 10, 20 can be in a range from about −70 nm to about 350 nm. In some aspects, a difference in SA between first and second orientations 10, 20 can be in a range from about −150 nm to about 0 nm. In some aspects, a difference in SA between first and second orientations 10, 20 can be in a range from about 0 nm to about 350 nm. In some aspects, a difference in SA between first and second orientations 10, 20 can be in a range from about −100 nm to about 100 nm. In some aspects, a difference in SA between first and second orientations 10, 20 can be in a range from about −50 nm to about 50 nm.

In some aspects, a difference in optical power between first and second orientations 10, 20 can be at least about 1 diopter. In some aspects, a difference in optical power between first and second orientations 10, 20 can be at least about −1 diopter. In some aspects, a difference in optical power between first and second orientations 10, 20 can be about 0.25 diopter. In some aspects, a difference in optical power between first and second orientations 10, 20 can be in a range from about −0.8 diopter to about 0.6 diopter. In some aspects, a difference in optical power between first and second orientations 10, 20 can be in a range from about −2.0 diopter to about 2.0 diopter. In some aspects, a difference in optical power between first and second orientations 10, 20 can be in a range from about −1.0 diopter to about 1.0 diopter. In some aspects, a difference in optical power between first and second orientations 10, 20 can be in a range from about −0.5 diopter to about 0.5 diopter. In some aspects, a difference in optical power between first and second orientations 10, 20 can be in a range from about −0.25 diopter to about 0.25 diopter.

FIGS. 5 and 5A illustrate reversible IOL 100′ in a first orientation 10 (forward), according to various example aspects. Reversible IOL 100′ can be configured to be reversible and provide two different SA corrections based on orientation (e.g., forward, backward). Reversible IOL 100′ can be further configured to have one or more aspheric profiles that contribute to the two different SA corrections based on orientation. Reversible IOL 100′ can be further configured to have one or more symmetric haptic devices (e.g., reflection symmetric) such that there is no change to the haptic devices based on orientation (e.g., forward, backward). Reversible IOL 100′ can be further configured to provide vaulted haptic devices that have a height extending away from a plane of the anterior and posterior optical elements.

Although reversible IOL 100′ is shown in FIGS. 5 and 5A as a stand-alone apparatus and/or system, the aspects of this disclosure can be used with other apparatuses, systems, and/or methods, such as, but not limited to, elements in FIGS. 1-4 and 6-8, e.g., reversible IOL 100, system 200, SA shift function 610, optical power shift function 710, and/or flow diagram 800.

The aspects of reversible IOL 100 shown in FIGS. 1, 1A, 2, and 2A, for example, and the aspects of reversible IOL 100′ shown in FIGS. 5 and 5A may be similar. Similar reference numbers are used to indicate features of the aspects of reversible IOL 100 shown in FIGS. 1, 1A, 2, and 2A and the similar features of the aspects of reversible IOL 100′ shown in FIGS. 5 and 5A. One difference between the aspects of reversible IOL 100 shown in FIGS. 1, 1A, 2, and 2A and the aspects of reversible IOL 100′ shown in FIGS. 5 and 5A is that reversible IOL 100′ includes anterior surface 112 with anterior aspheric surface 116, posterior surface 122 with posterior aspheric surface 126, and haptic device 140′ with vaults 148 extending a height 149 away from plane 146, rather than reversible IOL 100 with anterior surface 112, posterior surface 122, and haptic device 140 shown in FIGS. 1, 1A, 2, and 2A.

As shown in FIGS. 5 and 5A, reversible IOL 100′ can include anterior optical element 110 with anterior aspheric surface 116, posterior optical element 120 with posterior aspheric surface 126, and haptic device 140′. In some aspects, first SA correction 118 can be based at least in part on anterior aspheric surface 116. In some aspects, second SA correction 128 can be based at least in part on posterior aspheric surface 126. In some aspects, reversible IOL 100′ can include both anterior aspheric surface 116 and posterior aspheric surface 126, and produce first SA correction 118 in first orientation 10 and second SA correction 128 in second orientation 20.

Haptic device 140′ can be configured to secure and stabilize reversible IOL 100′ in place when implanted in an eye. Haptic device 140′ can be further configured to be arranged symmetrically about optical axis 102 of reversible IOL 100′ such that there is no change (e.g., reflection symmetric) of haptic device 140′ between first orientation 10 and second orientation 20. Haptic device 140′ can be further configured to provide a variable shift in height (vault) between first orientation 10 and second orientation 20.

As shown in FIGS. 5 and 5A, haptic device 140′ can include first haptic arm 142 with vault 148 and second haptic arm 144 with vault 148. Vaults 148 can be configured to vault or extend anterior optical element 110 and/or posterior optical element 120 a height 149 away from plane 146 depending on the orientation. In some aspects, for example, vaults 148 can extend away from plane 146 of anterior and posterior optical elements 110, 120 toward posterior optical element 120 in first orientation 10. In some aspects, for example, vaults 148 can extend away from plane 146 of anterior and posterior optical elements 110, 120 toward anterior optical element 110 in first orientation 10. In some aspects, first and second SA corrections 118, 128 can be based at least in part on height 149 of vaults 148.

### Example Coddington Shape Factor Dependence

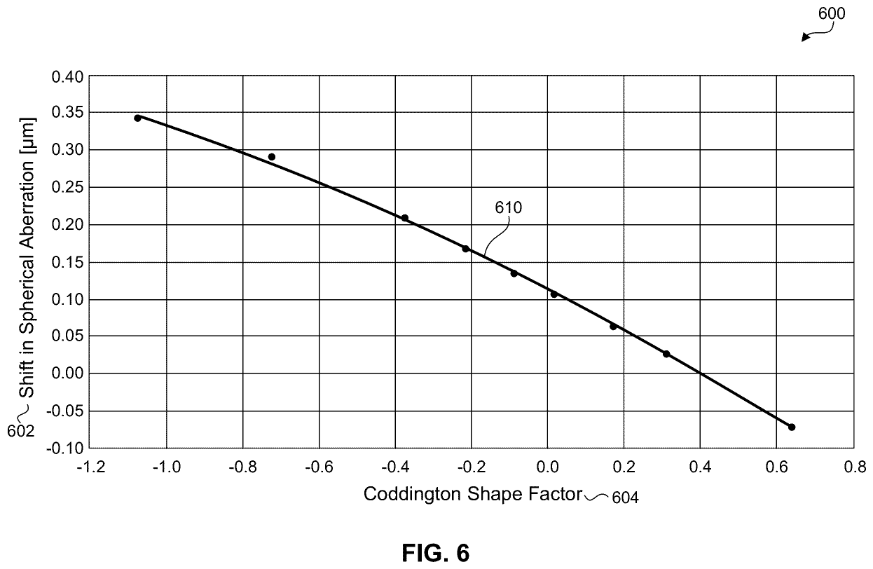

FIG. 6 shows plot 600 of SA shift function 610 of one or more reversible IOLs (e.g., reversible IOL 100, 100′), according to an example aspect. Although SA shift function 610 is shown in FIG. 6 as a stand-alone apparatus and/or system, the aspects of this disclosure can be used with other apparatuses, systems, and/or methods, such as, but not limited to, elements in FIGS. 1-5, 5A, 7, and 8, e.g., reversible IOL 100, system 200, reversible IOL 100′, optical power shift function 710, and/or flow diagram 800.

As shown in FIG. 6, plot 600 shows shifts in SA (μm) 602 (e.g., LSA) between first and second orientations 10, 20 of one or more reversible IOLs as a function of the Coddington shape factor 604. Plot 600 includes SA shift function 610 corresponding to how the SA changes between first orientation 10 (forward) and second orientation 20 (backward) for different Coddington shape factors. As shown in FIG. 6, as the Coddington shape factor 604 of the reversible IOL (e.g., reversible IOL 100, 100′) deviates from 0 (e.g., symmetric biconvex lens), the shift in SA (μm) 602 (e.g., LSA) becomes positive for negative Coddington shape factor and negative for positive Coddington shape factor. In some aspects, as the Coddington shape factor deviates from 0, a range of SA correction increases. In some aspects, as the Coddington shape factor becomes more negative, a higher corneal SA correction is corrected when the reversible IOL is flipped from first orientation 10 (forward) to second orientation 20 (backward). In some aspects, a single reversible IOL design (e.g., single Coddington shape factor, e.g., C₁=0.4=−C₂) can correct two different magnitudes of SA when flipped from first orientation 10 (forward) to second orientation 20 (backward).

In some aspects, a variable shift in SA between first and second orientations 10, 20 can be determined based on the Coddington shape factor of the reversible IOL (e.g., SA shift function 610). For example, a shift between first SA correction 118 and second SA correction 128 of reversible IOL 100, 100′ between first and second orientations 10, 20 can be determined (designed) based on the Coddington shape factor of reversible IOL 100, 100′ (e.g., C₁=0.4 and C₂=−0.4, corresponding to a SA shift of about 215 nm, etc.). These parameters are all examples for illustration purposes, and non-limiting.

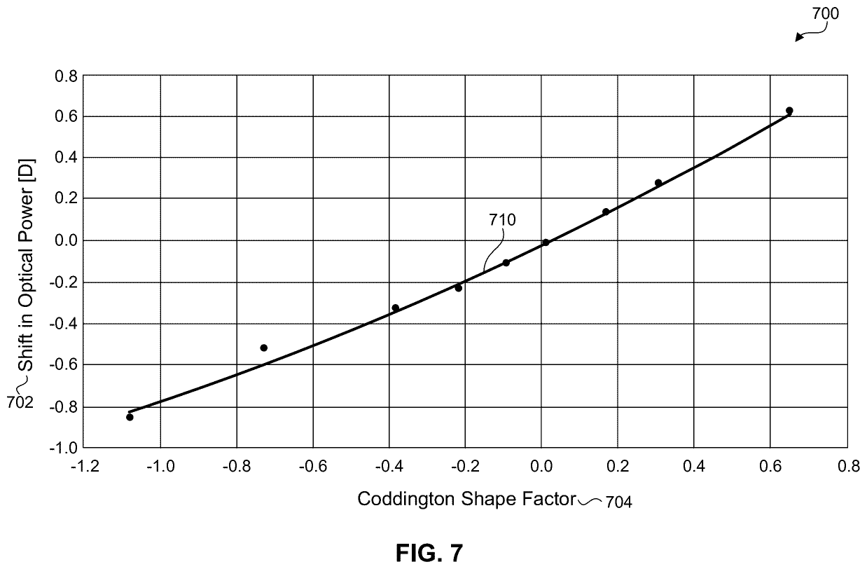

FIG. 7 shows plot 700 of optical power shift function 710 of one or more reversible IOLs (e.g., reversible IOL 100, 100′), according to an example aspect. Although optical power shift function 710 is shown in FIG. 7 as a stand-alone apparatus and/or system, the aspects of this disclosure can be used with other apparatuses, systems, and/or methods, such as, but not limited to, elements in FIGS. 1-5, 5A, 6, and 8, e.g., reversible IOL 100, system 200, reversible IOL 100′, SA shift function 610, and/or flow diagram 800.

As shown in FIG. 7, plot 700 shows shift in optical power (D) 702 between first and second orientations 10, 20 of one or more reversible IOLs as a function of the Coddington shape factor 704. Plot 700 includes optical power shift function 710 corresponding to how the optical power changes between first orientation 10 (forward) and second orientation 20 (backward) for different Coddington shape factors. As shown in FIG. 7, as the Coddington shape factor 704 of the reversible IOL (e.g., reversible IOL 100, 100′) deviates from 0 (e.g., symmetric biconvex lens), the shift in optical power (D) 702 becomes positive for positive Coddington shape factor and negative for negative Coddington shape factor. In some aspects, as the Coddington shape factor deviates from 0, a change in back focal length increases. In some aspects, as the Coddington shape factor becomes more negative, a dioptric shift becomes more negative when the reversible IOL is flipped from first orientation 10 (forward) to second orientation 20 (backward), indicating a drop in effective power of the reversible IOL. In some aspects, a single reversible IOL design (e.g., single Coddington shape factor, e.g., C₁=0.3=−C₂) can introduce a shift in effective power when flipped from first orientation 10 (forward) to second orientation 20 (backward).

In some aspects, a variable shift in optical power between first and second orientations 10, 20 can be determined based on the Coddington shape factor of the reversible IOL (e.g., optical power shift function 710). For example, a shift between a first shift in optical power (e.g., D₁=0.25 diopter) and a second shift in optical power (e.g., D₂=−0.27 diopter) of reversible IOL 100, 100′ from a baseline optical power (e.g., 20D) between first and second orientations 10, 20 can be determined (designed) based on the Coddington shape factor of reversible IOL 100, 100′ (e.g., C₁=0.3 and C₂=−0.3, corresponding to an optical power shift of about 0.52 diopter, etc.). In some aspects, reversible IOL 100, 100′ may have a first optical power (e.g., P₁=20D) in first orientation 10 and a second optical power (e.g., P₂=19.75D) in second orientation 20, corresponding to a shift in optical power of 0.25D (e.g., ΔP=P₁−P₂=(20D)−(19.75D)=0.25D) based on the Coddington shape factor. For example, as shown in FIG. 7, a shift in optical power (e.g., ΔP=0.25D) between first and second orientations 10, 20 may be determined (designed) based on the Coddington shaper factor of reversible IOL 100, 100′ with C₁=0.15 (e.g., corresponding to a first shift in optical power of D₁=0.12D) and C₂=−0.15 (e.g., corresponding to a second shift in optical power of D₂=−0.15D), corresponding to a shift in optical power of about 0.25D (e.g., absolute difference of |D₁−D₂|=ΔP=0.27D). In some aspects, reversible IOL 100, 100′ may have a first optical power (e.g., P₁=20D) in first orientation 10 and a second optical power (e.g., P₂=19.5D) in second orientation 20, corresponding to a shift in optical power of 0.5D (e.g., ΔP=P₁−P₂=(20D)−(19.5D)=0.5D) based on the Coddington shape factor. For example, as shown in FIG. 7, a shift in optical power (e.g., ΔP=0.5D) between first and second orientations 10, 20 may be determined (designed) based on the Coddington shaper factor of reversible IOL 100, 100′ with C₁=0.3 (e.g., corresponding to a first shift in optical power of D₁=0.25D) and C₂=−0.3 (e.g., corresponding to a second shift in optical power of D₂=−0.27D), corresponding to a shift in optical power of about 0.5D (e.g., absolute difference of |D₁−D₂|=ΔP=0.52D). In some aspects, reversible IOL 100, 100′ may have a first optical power (e.g., P₁=20D) in first orientation 10 and a second optical power (e.g., P₂=19D) in second orientation 20, corresponding to a shift in optical power of 1D (e.g., ΔP=P₁−P₂=(20D)−(19D)=1D) based on the Coddington shape factor. For example, as shown in FIG. 7, a shift in optical power (e.g., ΔP=1D) between first and second orientations 10, 20 may be determined (designed) based on the Coddington shaper factor of reversible IOL 100, 100′ with C₁=0.58 (e.g., corresponding to a first shift in optical power of D₁=0.54D) and C₂=−0.58 (e.g., corresponding to a second shift in optical power of D₂=−0.48D), corresponding to a shift in optical power of about 1D (e.g., absolute difference of |D₁−D₂|=ΔP=1.02D). These parameters are all examples for illustration purposes, and non-limiting.

In some aspects, a system can include a plurality of reversible IOLs (e.g., reversible IOL 100, 100′). In some aspects, each reversible IOL can include anterior optical element 110 configured to provide a first SA correction (e.g., first SA correction 118) and posterior optical element 120 configured to provide a second SA correction (e.g., second SA correction 128), where the first SA correction is different from the second SA correction.

In some aspects, the plurality of reversible IOLs (e.g., reversible IOL 100, 100′) can include a first reversible IOL and a second reversible IOL. In some aspects, the first reversible IOL can be configured to provide a first optical power. In some aspects, the second reversible IOL can be configured to provide a second optical power different from the first optical power.

In some aspects, for each reversible IOL, in first orientation 10 (forward), anterior optical element 110 can be configured to face cornea 152 of a user and, in second orientation 20 (backward), posterior optical element 120 can be configured to face cornea 152 of a user. In some aspects, a difference in optical power between first and second orientations 10, 20 for each reversible IOL can be about 0.25 diopter. In some aspects, each reversible IOL can be configured to provide a different difference in optical power between first and second orientations 10, 20 in a range from about −1.0 diopter to about 1.0 diopter. In some aspects, each reversible IOL can be configured to provide a different difference in SA between first and second orientations 10, 20 in a range from about −70 nm to about 350 nm.

### Example Flow Diagram

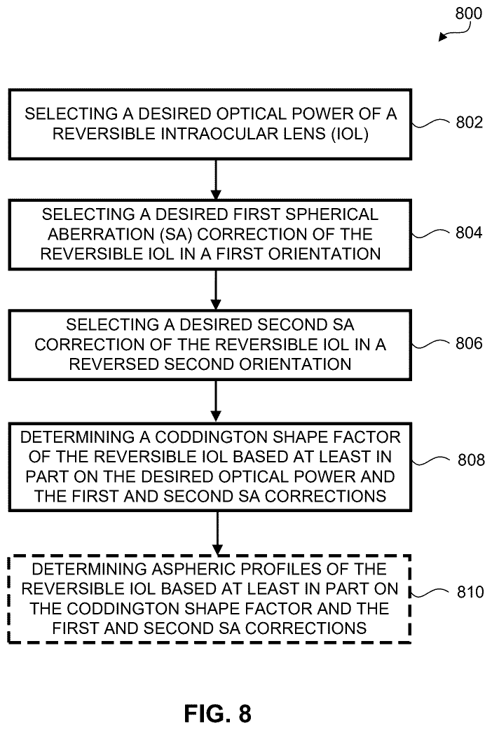

FIG. 8 illustrates flow diagram 800 according to an example aspect. For example, flow diagram 800 can be for reversible IOL 100 shown in FIGS. 1, 1A, 2, and 2A. Flow diagram 800 can be configured to design a reversible IOL with a desired optical power, a desired first SA correction in a first orientation (forward), and a desired second SA correction in a second orientation (backward). Flow diagram 800 can be further configured to determine a geometry (e.g., Coddington shape factor) of the reversible IOL to produce the desired optical power and desired first and second SA corrections. Flow diagram 800 can be further configured to determine aspheric profiles of the reversible IOL to produce the desired optical power and desired first and second SA corrections.

It is to be appreciated that not all operations in FIG. 8 are needed to perform the disclosure provided herein. Further, some of the operations may be performed simultaneously, sequentially, and/or in a different order than shown in FIG. 8. Flow diagram 800 shall be described with reference to FIGS. 1-7. However, flow diagram 800 is not limited to those example aspects. Although flow diagram 800 is shown in FIG. 8 as a stand-alone method, the aspects of this disclosure can be used with other apparatuses, systems, and/or methods, such as, but not limited to, elements in FIGS. 1-7, e.g., reversible IOL 100, system 200, reversible IOL 100′, SA shift function 610, and/or optical power shift function 710. In some aspects, flow diagram 800 can be implemented by one or more models or algorithms (e.g., optimization algorithm) run on one or more processors and/or computing devices based on one or more instructions stored in one or more memories.

In operation 802, as shown in the example of FIGS. 1-7, a desired optical power of reversible IOL 100 can be selected.

In operation 804, as shown in the example of FIGS. 1-7, a desired first SA correction of reversible IOL 100 in a first orientation 10 (forward) can be selected.

In operation 806, as shown in the example of FIGS. 1-7, a desired second SA correction of reversible IOL 100 in a second orientation 20 (backward) can be selected.

In operation 808, as shown in the example of FIGS. 1-7, a Coddington shape factor of reversible IOL 100 can be determined based at least in part on the desired optical power, the desired first SA correction, and the desired second SA correction. In some aspects, the Coddington shape factor can be determined based on an optimization model or optimization algorithm to optimize a set of parameters of reversible IOL 100 to produce the desired optical power and first and second SA corrections.

In some aspects, the parameters can include a desired optical power in first orientation 10, a desired optical power in second orientation 20, a desired first SA correction in first orientation 10, a desired second SA correction in second orientation 20, an anterior aspheric profile, a posterior aspheric profile, a focal length of anterior optical element 110, a focal length of posterior optical element 120, the anterior radius of curvature Rₐ 114 of anterior surface 112, the posterior radius of curvature Rₚ 124 of posterior surface 122, a height (vault) of haptic device 140 in first orientation 10, a height (vault) of haptic device 140 in second orientation 20, a SA shift function between first and second orientations 10, 20 (e.g., SA shift function 610), an optical power shift function between first and second orientations 10, 20 (e.g., optical power shift function 710), or a combination thereof.

In some aspects, the Coddington shape factor can be determined based on a model of reversible IOL 100 (e.g., ZEMAX) and the set of parameters. In some aspects, an inverse optimization of the set of parameters can be performed such that the model of reversible IOL 100 produces a desired optical power, desired first SA correction (e.g., first SA correction 118) in first orientation 10, and a desired second SA correction (e.g., second SA correction 128) in second orientation 20. In some aspects, the optimization model or optimization algorithm can perform simulated annealing, gradient descent, finite difference, interpolation, population models, regression, parameter adaptation, supervised machine learning, unsupervised machine learning, neural networks, classification models, clustering, vector quantization, stochastic gradient descent, implicit updates, leaky averaging, momentum methods, adaptive gradient (AdaGrad), backpropagation, root mean square propagation (RMSProp), adaptive moment estimation (Adam), or a combination thereof to determine the Coddington shape factor and/or one or more aspheric profiles of reversible IOL 100. In some aspects, the optimization model or optimization algorithm can perform gradient descent to determine the Coddington shape factor and/or one or more aspheric profiles of reversible IOL 100. In some aspects, the inverse optimization can be performed by an optimization algorithm (e.g., gradient descent).

In some aspects, determining the Coddington shape factor can include designing the reversible IOL, in first orientation 10, to provide an EDOF. In some aspects, determining the Coddington shape factor can include designing the reversible IOL, in second orientation 20, to provide an increased visual contrast at distance vision.

In operation 810, optionally, as shown in the example of FIGS. 1-7, one or more aspheric profiles of reversible IOL 100 can be determined based at least in part on the Coddington shape factor, the desired first SA correction, and the desired second SA correction. In some aspects, the one or more aspheric profiles of reversible IOL 100 can be determined based on the optimization model or optimization algorithm to optimize one or more aspheric surfaces of reversible IOL 100 (e.g., anterior aspheric surface 116, posterior aspheric surface 126) to produce the Coddington shape factor and desired first and second SA corrections.

In some aspects, flow diagram 800 can include determining a corneal SA based on one or more measured corneal spherical aberrations of a user or of a population. In some aspects, determining the Coddington shape factor can be based at least in part on a measured corneal SA of a user. In some aspects, determining the Coddington shape factor can be based at least in part on a mean distribution of measured corneal SA of a population (e.g., people with cataracts, people with LASIK surgery, people with astigmatism, etc.).

It is to be understood that the phraseology or terminology herein is for the purpose of description and not of limitation, such that the terminology or phraseology of the present specification is to be interpreted by those skilled in relevant art(s) in light of the teachings herein.

The above examples are illustrative, but not limiting, of the aspects of this disclosure. Other suitable modifications and adaptations of the variety of conditions and parameters normally encountered in the field, and which would be apparent to those skilled in the relevant art(s), are within the spirit and scope of the disclosure.

While specific aspects have been described above, it will be appreciated that the aspects may be practiced otherwise than as described. The description is not intended to limit the scope of the claims.

It is to be appreciated that the Detailed Description section, and not the Summary and Abstract sections, is intended to be used to interpret the claims. The Summary and Abstract sections may set forth one or more but not all example aspects as contemplated by the inventor(s), and thus, are not intended to limit the aspects and the appended claims in any way.

The aspects have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the aspects that others can, by applying knowledge within the skill of the art, readily modify and/or adapt for various applications such specific aspects, without undue experimentation, without departing from the general concept of the aspects. Therefore, such adaptations and modifications are intended to be within the meaning and range of equivalents of the disclosed aspects, based on the teaching and guidance presented herein.

The breadth and scope of the aspects should not be limited by any of the above-described example aspects, but should be defined only in accordance with the following claims and their equivalents.

The present disclosure may also be described in accordance with the following clauses:

Clause 1. A reversible ophthalmic lens comprising:
an anterior optical surface configured to face a cornea of a user in a first orientation of the reversible ophthalmic lens; and
a posterior optical surface configured to face a cornea of a user in a second orientation of the reversible ophthalmic lens,
wherein the reversible ophthalmic lens provides a first spherical aberration correction in the first orientation and a second spherical aberration correction in the second orientation, and
wherein the first spherical aberration correction is different from the second spherical aberration correction.

Clause 2. The reversible ophthalmic lens of clause 1, wherein the first and second spherical aberration corrections are based at least in part on a Coddington shape factor:

$$C = \frac{{R}_{p} + {R}_{a}}{{R}_{p} - {R}_{a}}$$

where Rₐ is a radius of curvature of the anterior optical surface and Rₚ is a radius of curvature of the posterior optical surface.

Clause 3. The reversible ophthalmic lens of clause 2, wherein the Coddington shape factor is in a range from −1.2 to 1.2.

Clause 4. The reversible ophthalmic lens of any one of clauses 1 to 3, wherein the first and second spherical aberration corrections are predetermined and based at least in part on a desired optical power.

Clause 5. The reversible ophthalmic lens of any one of clauses 1 to 4, wherein the first and second spherical aberration corrections are based at least in part on a population distribution of measured corneal spherical aberration data.

Clause 6. The reversible ophthalmic lens of any one of clauses 1 to 5, wherein:
in the first orientation, the first spherical aberration correction is configured to provide a first optical power, and
in the second orientation, the second spherical aberration correction is configured to provide the first optical power.

Clause 7. The reversible ophthalmic lens of any one of clauses 1 to 6, wherein:
in the first orientation, the first spherical aberration correction is configured to provide an extended depth of focus (EDOF), and
in the second orientation, the second spherical aberration correction is configured to provide a different EDOF or an increased visual contrast at distance vision.

Clause 8. The reversible ophthalmic lens of any one of clauses 1 to 7, wherein:
in the first orientation, the first spherical aberration correction is configured to provide a first EDOF or a first visual contrast at distance vision, and
in the second orientation, the second spherical aberration correction is configured to provide a second EDOF different from the first EDOF or a second visual contrast at distance vision different from the first visual contrast at distance vision.

Clause 9. The reversible ophthalmic lens of any one of clauses 1 to 8, wherein a difference in spherical aberration between the first and second orientations is at least 250 nm.

Clause 10. The reversible ophthalmic lens of any one of clauses 1 to 8, wherein a difference in spherical aberration between the first and second orientations is in a range from −70 nm to 350 nm.

Clause 11. The reversible ophthalmic lens of any one of clauses 1 to 10, wherein a difference in optical power between the first and second orientations is at least 1 diopter.

Clause 12. The reversible ophthalmic lens of any one of clauses 1 to 10, wherein a difference in optical power between the first and second orientations is 0.25 diopter.

Clause 13. The reversible ophthalmic lens of any one of clauses 1 to 10, wherein a difference in optical power between the first and second orientations is in a range from −0.8 diopter to 0.6 diopter.

Clause 14. The reversible ophthalmic lens of any one of clauses 1 to 13, wherein:
the anterior optical surface comprises an anterior aspheric surface, and
the first spherical aberration correction is based at least in part on the anterior aspheric surface.

Clause 15. The reversible ophthalmic lens of any one of clauses 1 to 14, wherein:
the posterior optical surface comprises a posterior aspheric surface, and
the second spherical aberration correction is based at least in part on the posterior aspheric surface.

Clause 16. The reversible ophthalmic lens of any one of clauses 1 to 15, wherein the anterior and posterior optical surfaces are part of a unitary optic.

Clause 17. The reversible ophthalmic lens of any one of clauses 1 to 15, wherein:
the anterior optical surface is part of a monofocal lens; and
the posterior optical surface is part of a monofocal lens.

Clause 18. The reversible ophthalmic lens of any one of clauses 1 to 15, wherein:
the anterior optical surface is part of a multifocal lens; and
the posterior optical surface is part of a multifocal lens.

Clause 19. The reversible ophthalmic lens of any one of clauses 1 to 18, wherein the anterior and posterior optical surfaces comprise a flexible material, a foldable material, a shape memory material, or a combination thereof.

Clause 20. The reversible ophthalmic lens of any one of clauses 1 to 19, further comprising one or more haptic devices coupled to the anterior optical surface, the posterior optical surface, or both.

Clause 21. The reversible ophthalmic lens of clause 20, wherein the one or more haptic devices is configured to extend in a plane of the anterior and posterior optical surfaces.

Clause 22. The reversible ophthalmic lens of clause 20 or clause 21, wherein the one or more haptic devices comprises one or more vaults configured to extend out of a plane of the anterior and posterior optical surfaces.

Clause 23. The reversible ophthalmic lens of clause 22, wherein the first and second spherical aberration corrections are based at least in part on a height of the one or more vaults.

Clause 24. The reversible ophthalmic lens of any one of clauses 20 to 23, wherein the one or more haptic devices are arranged symmetrically about an optical axis of the reversible ophthalmic lens.

Clause 25. A system comprising:
a plurality of reversible ophthalmic lenses,
wherein each reversible ophthalmic lens is a reversible ophthalmic lens of any one of clauses 1 to 24.

Clause 26. The system of clause 25, wherein:
the plurality of reversible ophthalmic lens comprises a first reversible ophthalmic lens and a second reversible ophthalmic lens,
the first reversible ophthalmic lens configured to provide a first optical power, and
the second reversible ophthalmic lens configured to provide a second optical power different from the first optical power.

Clause 27. The system of clause 25 or clause 26, wherein a difference in optical power between the first and second orientations for each reversible ophthalmic lens is 0.25 diopter.

Clause 28. The system of any one of clauses 25 to 27, wherein each reversible ophthalmic lens is configured to provide a different difference in optical power between the first and second orientations in a range from −1 diopter to 1 diopter.

Clause 29. The system of any one of clauses 25 to 28, wherein each reversible ophthalmic lens is configured to provide a different difference in spherical aberration between the first and second orientations in a range from −70 nm to 350 nm.

Clause 30. A method of designing a reversible ophthalmic lens, the method comprising:
selecting a desired optical power of the reversible ophthalmic lens;
selecting a desired first spherical aberration correction of the reversible ophthalmic lens in a first orientation in which an anterior optical element of the reversible ophthalmic lens is configured to face a cornea of a user;
selecting a desired second spherical aberration correction of the reversible ophthalmic lens in a second orientation in which a posterior optical element of the reversible ophthalmic lens is configured to face a cornea of a user; and
determining a Coddington shape factor of the reversible ophthalmic lens based at least in part on the desired optical power, the desired first spherical aberration correction, and the desired second spherical aberration correction.

Clause 31. The method of clause 30, further comprising determining one or more aspheric profiles of the reversible ophthalmic lens based at least in part on the Coddington shape factor, the desired first spherical aberration correction, and the desired second spherical aberration correction.

Clause 32. The method of clause 30 or clause 31, wherein determining the Coddington shape factor comprises designing the reversible ophthalmic lens, in the first orientation, to provide an extended depth of focus (EDOF).

Clause 33. The method of any one of clauses 30 to 32, wherein determining the Coddington shape factor comprises designing the reversible ophthalmic lens, in the second orientation, to provide an increased visual contrast at distance vision.

Clause 34. The method of any one of clauses 30 to 33, further comprising determining a corneal spherical aberration based on one or more measured corneal spherical aberrations of a user or of a population.

Clause 35. A reversible ophthalmic lens comprising:
an anterior optical surface configured to face a cornea of a user in a first orientation of the reversible ophthalmic lens; and
a posterior optical surface configured to face a cornea of a user in a second orientation of the reversible ophthalmic lens,
wherein the reversible ophthalmic lens provides a first optical power in the first orientation and a second optical power in the second orientation, and
wherein the first optical power is different from the second optical power by a desired shift in optical power.

Clause 36. The reversible ophthalmic lens of clause 35, wherein the desired shift in optical power, first optical power, and second optical power are based at least in part on a Coddington shape factor:

$$C = \frac{{R}_{p} + {R}_{a}}{{R}_{p} - {R}_{a}}$$

where Rₐ is a radius of curvature of the anterior optical surface and Rₚ is a radius of curvature of the posterior optical surface.

Clause 37. The reversible ophthalmic lens of clause 36, wherein the Coddington shape factor is in a range from −1.2 to 1.2.

Clause 38. The reversible ophthalmic lens of any one of clauses 35 to 37, wherein the first and second optical powers are predetermined and based at least in part on the desired shift in optical power.

Clause 39. The reversible ophthalmic lens of any one of clauses 35 to 38, wherein the desired shift in optical power between the first and second orientations is at least 1 diopter.

Clause 40. The reversible ophthalmic lens of any one of clauses 35 to 38, wherein the desired shift in optical power between the first and second orientations is 0.25 diopter.

Clause 41. The reversible ophthalmic lens of any one of clauses 35 to 38, wherein the desired shift in optical power between the first and second orientations is in a range from −0.8 diopter to 0.6 diopter.

Clause 42. The reversible ophthalmic lens of any one of clauses 35 to 41, wherein the anterior and posterior optical surfaces are part of a unitary optic.

Clause 43. The reversible ophthalmic lens of any one of clauses 35 to 41, wherein:
the anterior optical surface is part of a monofocal lens; and
the posterior optical surface is part of a monofocal lens.

Clause 44. The reversible ophthalmic lens of any one of clauses 35 to 41, wherein:
the anterior optical surface is part of a multifocal lens; and
the posterior optical surface is part of a multifocal lens.

Clause 45. The reversible ophthalmic lens of any one of clauses 35 to 44, wherein the anterior and posterior optical surfaces comprise a flexible material, a foldable material, a shape memory material, or a combination thereof.

Clause 46. The reversible ophthalmic lens of any one of clauses 35 to 45, further comprising one or more haptic devices coupled to the anterior optical surface, the posterior optical surface, or both.

Clause 47. A system comprising:
a plurality of reversible ophthalmic lenses,
wherein each reversible ophthalmic lens is a reversible ophthalmic lens of any one of clauses 35 to 46.

Clause 48. The system of clause 47, wherein:
the plurality of reversible ophthalmic lens comprises a first reversible ophthalmic lens and a second reversible ophthalmic lens,
the first reversible ophthalmic lens configured to provide a first optical power, and
the second reversible ophthalmic lens configured to provide a second optical power different from the first optical power.

Clause 49. The system of clause 47 or clause 48, wherein a difference in optical power between the first and second orientations for each reversible ophthalmic lens is 0.25 diopter.

Clause 50. The system of any one of clauses 47 to 49, wherein each reversible ophthalmic lens is configured to provide a different difference in optical power between the first and second orientations in a range from −1 diopter to 1 diopter.

Clause 51. A method of designing a reversible ophthalmic lens, the method comprising:
selecting a desired shift in optical power of the reversible ophthalmic lens;
selecting a desired first optical power of the reversible ophthalmic lens in a first orientation in which an anterior optical element of the reversible ophthalmic lens is configured to face a cornea of a user;
selecting a desired second optical power of the reversible ophthalmic lens in a second orientation in which a posterior optical element of the reversible ophthalmic lens is configured to face a cornea of a user; and
determining a Coddington shape factor of the reversible ophthalmic lens based at least in part on the desired shift in optical power, the desired first optical power, and the desired second spherical optical power.

Clause 52. The method of clause 51, further comprising determining one or more aspheric profiles of the reversible ophthalmic lens based at least in part on the Coddington shape factor and the desired shift in optical power.

Clause 53. The method of clause 51 or clause 52, wherein determining the Coddington shape factor comprises designing the reversible ophthalmic lens, in the first orientation, to provide an extended depth of focus (EDOF).

Clause 54. The method of any one of clauses 51 to 53, wherein determining the Coddington shape factor comprises designing the reversible ophthalmic lens, in the second orientation, to provide an increased visual contrast at distance vision.

What is claimed is:

1. A reversible ophthalmic lens comprising:
an anterior optical surface configured to face a cornea of a user in a first orientation of the reversible ophthalmic lens; and
a posterior optical surface configured to face a cornea of a user in a second orientation of the reversible ophthalmic lens,
wherein the reversible ophthalmic lens provides a first optical power in the first orientation and a second optical power in the second orientation, and
wherein the first optical power is different from the second optical power by a desired shift in optical power.

2. The reversible ophthalmic lens of claim 1, wherein the desired shift in optical power, first optical power, and second optical power are based at least in part on a Coddington shape factor:
$$C = \frac{{R}_{p} + {R}_{a}}{{R}_{p} - {R}_{a}}$$
where Rₐ is a radius of curvature of the anterior optical surface and Rₚ is a radius of curvature of the posterior optical surface.

3. The reversible ophthalmic lens of claim 2, wherein the Coddington shape factor is in a range from −1.2 to 1.2.

4. The reversible ophthalmic lens of claim 1, wherein the first and second optical powers are predetermined and based at least in part on the desired shift in optical power.

5. The reversible ophthalmic lens of claim 1, wherein the desired shift in optical power between the first and second orientations is at least 1 diopter.

6. The reversible ophthalmic lens of claim 1, wherein the desired shift in optical power between the first and second orientations is 0.25 diopter.

7. The reversible ophthalmic lens of claim 1, wherein the desired shift in optical power between the first and second orientations is in a range from −0.8 diopter to 0.6 diopter.

8. The reversible ophthalmic lens of claim 1, wherein the anterior and posterior optical surfaces are part of a unitary optic.

9. The reversible ophthalmic lens of claim 1, wherein:
the anterior optical surface is part of a monofocal lens; and
the posterior optical surface is part of a monofocal lens.

10. The reversible ophthalmic lens of claim 1, wherein:
the anterior optical surface is part of a multifocal lens; and
the posterior optical surface is part of a multifocal lens.

11. The reversible ophthalmic lens of claim 1, wherein the anterior and posterior optical surfaces comprise a flexible material, a foldable material, a shape memory material, or a combination thereof.

12. The reversible ophthalmic lens of claim 1, further comprising one or more haptic devices coupled to the anterior optical surface, the posterior optical surface, or both.

13. A system comprising:
a plurality of reversible ophthalmic lenses,
wherein each reversible ophthalmic lens is a reversible ophthalmic lens of claim 1.

14. The system of claim 13, wherein:
the plurality of reversible ophthalmic lens comprises a first reversible ophthalmic lens and a second reversible ophthalmic lens,
the first reversible ophthalmic lens configured to provide a first optical power, and
the second reversible ophthalmic lens configured to provide a second optical power different from the first optical power.

15. The system of claim 13, wherein a difference in optical power between the first and second orientations for each reversible ophthalmic lens is 0.25 diopter.

16. The system of claim 13, wherein each reversible ophthalmic lens is configured to provide a different difference in optical power between the first and second orientations in a range from −1 diopter to 1 diopter.

17. A method of designing a reversible ophthalmic lens, the method comprising:
selecting a desired shift in optical power of the reversible ophthalmic lens;
selecting a desired first optical power of the reversible ophthalmic lens in a first orientation in which an anterior optical element of the reversible ophthalmic lens is configured to face a cornea of a user;
selecting a desired second optical power of the reversible ophthalmic lens in a second orientation in which a posterior optical element of the reversible ophthalmic lens is configured to face a cornea of a user; and
determining a Coddington shape factor of the reversible ophthalmic lens based at least in part on the desired shift in optical power, the desired first optical power, and the desired second spherical optical power.

18. The method of claim 17, further comprising determining one or more aspheric profiles of the reversible ophthalmic lens based at least in part on the Coddington shape factor and the desired shift in optical power.

19. The method of claim 17, wherein determining the Coddington shape factor comprises designing the reversible ophthalmic lens, in the first orientation, to provide an extended depth of focus (EDOF).

20. The method of claim 17, wherein determining the Coddington shape factor comprises designing the reversible ophthalmic lens, in the second orientation, to provide an increased visual contrast at distance vision.
